(19) United States
(12) Patent Application Publication
(10) Pub. No.: US 2013/0226271 A1
(43) Pub. Date: Aug. 29, 2013

(54) METHOD AND APPARATUS TO PROMOTE INFLAMMATION IN SPINAL TISSUES

(71) Applicant: Bret A. Ferree, Cincinnati, OH (US)

(72) Inventor: Bret A. Ferree, Cincinnati, OH (US)

(21) Appl. No.: 13/859,965

(22) Filed: Apr. 10, 2013

Related U.S. Application Data
(63) Continuation-in-part of application No. 12/855,971, filed on Aug. 13, 2010.
(60) Provisional application No. 61/622,311, filed on Apr. 10, 2012.

(51) Int. Cl. A61F 7/00

(52) U.S. Cl. CPC A61F 7/00
USPC 607/96

(57) ABSTRACT

Apparatus for treating tissue, wherein the apparatus heats the tissue in a controlled manner so as to promote therapeutic inflammation in the tissue, whereby to augment healing of the tissue.

### REFERENCE TO PENDING PRIOR PATENT APPLICATIONS

This patent application is:

(i) a continuation-in-part of pending prior U.S. patent application Ser. No. 12/855,971, filed Aug. 13, 2010 by Bret A. Ferree for METHOD AND APPARATUS FOR REPAIRING AND/OR REPLACING INTERVERTEBRAL DISC COMPONENTS AND PROMOTING HEALING (Attorney's Docket No. FERREE-BAF-23002/29); and

(ii) claims benefit of pending prior U.S. Provisional Patent Application Ser. No. 61/622,311, filed Apr. 10, 2012 by Bret A. Ferree for METHOD AND APPARATUS TO PROMOTE INFLAMMATION IN SPINAL TISSUES (Attorney's Docket No. FERREE-6 PROV).

The two (2) above-identified patent applications are hereby incorporated herein by reference.

### FIELD OF THE INVENTION

This invention relates generally to the treatment of spinal conditions such as deformity, fractures, intervertebral disc herniations, infections, tumors, and degenerative disc disease, and, in particular, to apparatus and methods for promoting inflammation in spinal tissues, such as vertebrae and intervertebral discs.

### BACKGROUND OF THE INVENTION

The human intervertebral disc is an oval to kidney bean-shaped structure of variable size depending on the location in the spine. The outer portion of the disc is known as the anulus fibrosus (AF, also known as the “anulus fibrosis”, or simply “the annulus”). The anulus fibrosus (AF) is made of ten to twenty collagen fiber lamellae. The collagen fibers within a lamella are parallel. Successive lamellae are oriented in alternating directions. About 48 percent of the lamellae are incomplete, but this value varies based upon location and increases with age. On average, the lamellae lie at an angle of sixty degrees with respect to the vertebral axis line, but this too varies depending upon location. The orientation serves to control vertebral motion (one half of the bands tighten to check motion when the vertebra above or below the disc are turned in either direction).

The anulus fibrosus contains the nucleus pulposus (NP, or simply “the nucleus”). The nucleus pulposus serves to transmit and dampen axial loads. A high water content (approximately 70-80 percent) assists the nucleus in this function. The water content has a diurnal variation. The nucleus imbibes water while a person lies recumbent. Nuclear material removed from the body and placed into water will imbibe water, swelling to several times its normal size. Activity squeezes fluid from the disc. The nucleus comprises roughly 50 percent of the entire disc. The nucleus contains cells (chondrocytes and fibrocytes) and proteoglycans (chondroitin sulfate and keratin sulfate). The cell density in the nucleus is on the order of 4,000 cells per microliter.

The intervertebral disc changes or “degenerates” with age. As a person ages, the water content of the disc falls from approximately 85 percent at birth to approximately 70 percent in the elderly. The ratio of chondroitin sulfate to keratin sulfate decreases with age, while the ratio of chondroitin 6 sulfate to chondroitin 4 sulfate increases with age. The distinction between the anulus and the nucleus decreases with age. Generally disc degeneration is painless.

Premature or accelerated disc degeneration is known as degenerative disc disease. A large portion of patients suffering from chronic low back pain are thought to have this condition. As the disc degenerates, the nucleus and anulus functions are compromised. The nucleus becomes thinner and less able to handle compression loads. The anulus fibers become redundant as the nucleus shrinks. The redundant anular fibers are less effective in controlling vertebral motion. This disc pathology can result in: 1) bulging of the anulus into the spinal cord or nerves; 2) narrowing of the space between the vertebra where the nerves exit; 3) tears of the anulus as abnormal loads are transmitted to the anulus and the anulus is subjected to excessive motion between vertebra; and 4) disc herniation or extrusion of the nucleus through complete anular tears.

Current surgical treatments for disc degeneration are destructive. One group of procedures, which includes lumbar discectomy, removes the nucleus or a portion of the nucleus. A second group of procedures destroy nuclear material. This group includes Chymopapin (an enzyme) injection, laser discectomy, and thermal therapy (heat treatment to denature proteins in the nucleus, whereby to destroy nuclear material). The first two groups of procedures compromise the treated disc. A third group, which includes spinal fusion procedures, either removes the disc or the disc's function by connecting two or more vertebra together, e.g., with fused bone. Fusion procedures transmit additional stress to the adjacent discs, which results in premature disc degeneration of the adjacent discs. These destructive procedures lead to acceleration of disc degeneration.

Prosthetic disc replacement offers many advantages. The prosthetic disc attempts to eliminate a patient's pain while preserving the disc's function. Current prosthetic disc implants either replace the nucleus or replace both the nucleus and the anulus. Both types of current procedures remove the degenerated disc component to allow room for the prosthetic component. Although the use of resilient materials has been proposed, the need remains for further improvements in the way in which prosthetic components are incorporated into the disc space to ensure strength and longevity. Such improvements are necessary, since the prosthesis may be subjected to 100,000,000 compression cycles over the life of the implant.

Current nucleus replacements (NRs) may cause lower back pain if too much pressure is applied to the anulus fibrosus. As discussed in U.S. Pat. Nos. 6,878,167 and 7,201,774, the content of each being expressly incorporated herein by reference in their entirety, the posterior portion of the anulus fibrosus has abundant pain fibers.

Herniated nucleus pulposus (HNP) occurs from tears in the anulus fibrosus. The herniated nucleus pulposus often applies pressure on the nerves or spinal cord. Compressed nerves cause back and leg or arm pain. Although a patient's symptoms result primarily from pressure applied by the bulging nucleus pulposus, the primary pathology lies in the anulus fibrosus.

Surgery for herniated nucleus pulposus, known as microlumbar discectomy (MLD), only addresses the nucleus pulposus. The opening in the anulus fibrosus is enlarged during surgery, further weakening the anulus fibrosus. Surgeons also remove generous amounts of the nucleus pulposus to reduce the risk of extruding additional portions of nucleus pulposus through the defect in the anulus fibrosus. Although microlumbar discectomy decreases or eliminates a patient's leg or arm pain, the procedure further damages already-weakened discs, since the initial opening in the annulus is enlarged during MLD surgery. Twenty-eight percent of patients seek additional medical or surgical treatment for back or leg pain within eighteen months following lumbar discectomy. Twenty-five percent of patients undergo reoperation within ten years following lumbar discectomy. Twenty-five percent of patients experience adjacent level disc degeneration following cervical disc surgery.

### SUMMARY OF THE INVENTION

The invention, broadly described, heats in a controlled manner spinal tissues such as the intervertebral disc (IVD), vertebrae, spinal ligaments, and muscles and other tissues surrounding the spine, to promote inflammation in such tissues, which augments healing of those tissues. The invention may also be used in the treatment of spinal conditions such as herniated discs, disc degeneration, deformities, fractures, tumors, infections, and pseudoarthrosis. The method and apparatus may be used to treat discs throughout the spine including the cervical, thoracic, and lumbar spines of humans and animals. For example, the method and apparatus may be used in surgeries on the anterior, lateral, or posterior portions of the spine. The method and apparatus may be used in other bones or tissues of the body. For example, the invention may be used to treat meniscus, cartilage, ligament and bone injury or degeneration of the knee, labrum, ligament, cartilage, or bone injury or degeneration of the hip or shoulder, muscle, ligament, tendon, bone, or cartilage injury or degeneration of other parts of the body, and injuries or disorders of other tissues of the body such as organs, blood vessels, or nerves. The invention may be used to treat chronic inflammatory conditions and autoimmune diseases such as rheumatoid arthritis, systemic lupus erythematosus, diabetes mellitus type 1, Crohns disease, Graves' disease, Hashimoto's thyroiditis, and Wegener's granulomatosis. The controlled heating of the present invention attracts neutrophils and especially macrophages to such target tissues, promoting the normal healing inflammatory process, which at its conclusion recruits reparative cells, such as fibrocytes, and drives away inflammatory cells, especially lymphocytes.

The invention preferably heats body tissues with living cells to 38° C. to 41.9° C. for a period of several hours to two or more weeks, more preferably heats body tissues with living cells to 39° C. to 41.9° C. for a period of ten hours to one week, and most preferably heats body tissues with living cells to 40° C. to 41° C. for a period of one to three days. In certain preferred embodiments of the invention, tissues without living cells, including allograft tissue, or synthetic materials, such as bone growth material including allograft tissue, hydroxyapatite (ProOsteon, Biomet, Parsippany, N.J.), calcium sulfate (Osteoset, Wright Medical technology, Arlington, Tenn.), demineralized bone matrix (AlloGro, AlloSource, Centennial, Colo.), collagen-based matrices, calcium phosphate, and bioglass, are preferably heated higher than 40° C. to 42° C. in order to heat the living tissues surrounding such allograft or synthetic materials to the previously mentioned preferred temperatures, which are preferably less than 42° C., whereby to promote the normal healing inflammatory process in those surrounding living tissues.

Autogenous bone graft contains living cells capable of synthesizing new bone (osteogenesis), growth factors (osteoinduction) and a structural matrix that acts as a scaffold (osteoconduction). Many autograft bone graft substitutes, such as hydroxyapatite, collagen-based matrices, calcium phosphate, calcium sulfate, and bioactive glass, are osteoconductive only. Patients' cells must populate such bone graft materials to successfully build bone. The present invention, by promoting inflammation, attracts patients' osteogenic cells to the bone growth promoting materials.

Patients' macrophages, a type of leukocyte, generally phagocytize and remove most herniated nucleus pulposus (HNP) tissue, which alleviates patients' symptoms. Unfortunately, the aforementioned natural macrophage process is inadequate for some patients, e.g., the more than 450,000 US patients who annually undergo cervical, thoracic, or lumbar discectomy. The present invention, by promoting inflammation with controlled heating of living tissue, attracts patients' macrophages to HNP tissue. Such embodiment of the invention augments the natural macrophage-mediated HNP removal, which helps patients recover from HNP without surgery.

Macrophages, more specifically activated macrophages, are required for wound healing. The present invention, by controlled heating of living tissue, recruits macrophages to injured tissue and activates them to optimize tissue healing.

In one preferred form of the invention, there is provided apparatus for treating tissue, wherein the apparatus heats the tissue in a controlled manner so as to promote therapeutic inflammation in the tissue, whereby to augment healing of the tissue.

In another preferred form of the invention, there is provided a method for treating tissue, comprising:

heating the tissue in a controlled manner so as to promote therapeutic inflammation in the tissue, whereby to augment healing of the tissue.

### BRIEF DESCRIPTION OF THE DRAWINGS

FIG. 1A is a posterior view of a portion of the spine, bone growth material and a preferred embodiment of the invention;

FIG. 1B is a superior view of a partial transverse cross-section of the spinal segment, bone growth material, and the embodiment of the invention shown in FIG. 1A;

FIG. 2A is a posterior view of a portion of the spine, bone growth material and an alternative embodiment of the invention;

FIG. 2B is a superior view of a partial transverse cross-section of the spinal segment, bone growth material, and the alternative embodiment of the invention shown in FIG. 2A;

FIG. 3A is an anterior view of a portion of the spine, an intradiscal fusion device, and another alternative embodiment of the invention;

FIG. 3B is a superior view of a partial transverse cross-section of the spinal segment, bone growth material, intradiscal fusion device and the alternative embodiment of the invention shown in FIG. 3A;

FIG. 4 is an anterior view of a partial coronal cross-section of a spinal segment, intradiscal device, bone growth material and an alternative embodiment of the invention;

FIG. 5 is an anterior view of a partial coronal cross-section of a spinal segment, intradiscal device, bone growth material and another alternative embodiment of the invention;

FIG. 6 is a superior view of a transverse cross-section of the spinal segment, bone growth material, and another alternative embodiment of the invention;

FIG. 7 is a posterior view of a spinal segment, bone growth material, and another alternative embodiment of the invention;

FIG. 8A is a lateral view of a spinal segment and another alternative embodiment of the invention;

FIG. 8B is a lateral view of a partial sagittal cross-section of the spinal segment and the embodiment of the invention shown in FIG. 8A;

FIG. 9A is a lateral view of a spinal segment and another alternative embodiment of the invention;

FIG. 9B is a lateral view of a partial sagittal cross-section of the spinal segment and the embodiment of the invention shown in FIG. 9A;

FIG. 10A is a superior view of a partial transverse cross-section of a spinal segment and another alternative embodiment of the invention;

FIG. 10B is a superior view of a partial transverse cross-section of a spinal segment and another alternative embodiment of the invention;

FIG. 10C is a superior view of a partial transverse cross-section of a spinal segment and another alternative embodiment of the invention;

FIG. 11A is a lateral view of a spinal segment and another alternative embodiment of the invention;

FIG. 11B is a lateral view of a partial sagittal cross-section of the spinal segment and the embodiment of the invention shown in FIG. 11A;

FIG. 12 is a lateral view of a spinal segment and another alternative embodiment of the invention;

FIG. 13A is a superior view of the distal end of an alternative embodiment of the invention;

FIG. 13B is a lateral view of additional apparatus used in conjunction with the embodiment of the invention shown in FIG. 13A;

FIG. 13C is a superior view of a partial transverse cross-section of a spinal segment and the embodiments of the invention shown in FIGS. 13A & B; and

FIG. 14 is a superior view of a partial transverse cross-section of human body, including a spinal segment, and the embodiment of the invention shown in FIG. 13C.

### DETAILED DESCRIPTION DESCRIPTION OF THE INVENTION

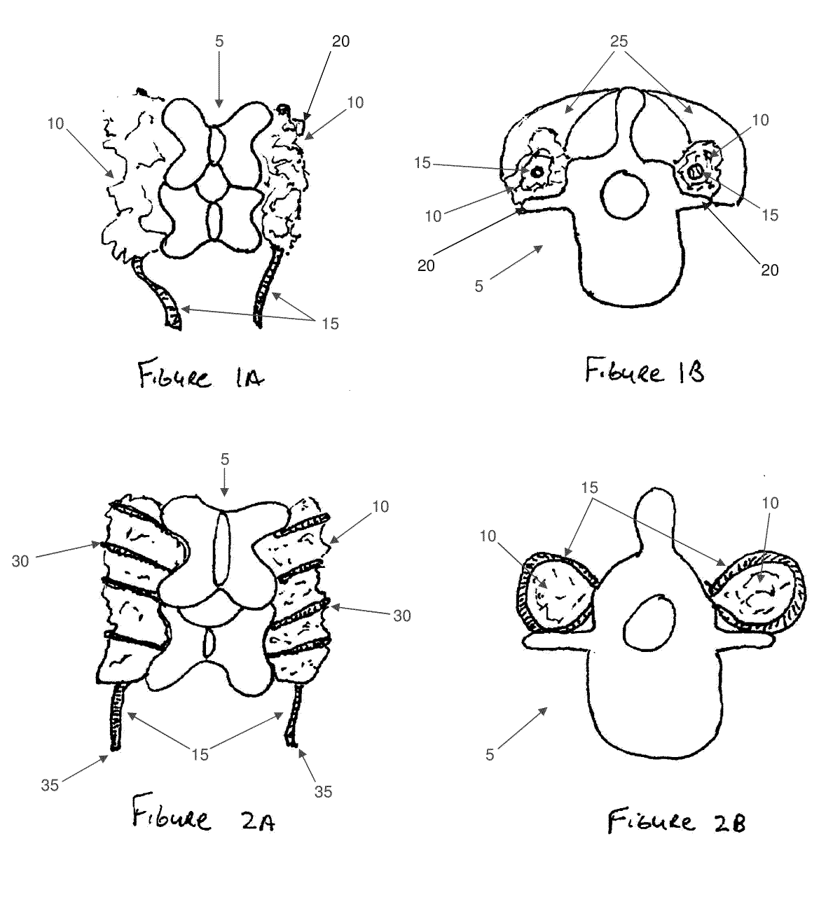

FIG. 1A is a posterior view of a portion of the spine 5, bone growth material 10 and a preferred embodiment of the invention. Two heating elements 15 are seen coursing through and from bone growth material 10 that lies over the transverse processes 20 of two adjacent vertebrae. The bone growth material 10 can be transplanted autograft bone from the patient, or preferably allograft bone or demineralized bone matrix (AlloGro, AlloSource, Centennial, Colo.) from donors, or most preferably a synthetic bone substitute, such as hydroxyapatite (ProOsteon, Biomet, Parsippany, N.J.), collagen-based matrices, calcium phosphate, calcium sulfate (Osteoset, Wright Medical technology, Arlington, Tenn.), and bioactive glass. The heat emitting portions of the heating elements 15 are preferably limited to the portion of the elements which are located within the area to be fused or a few millimeters or centimeters beyond that area. Such portions of the elements are preferably about 4 to 6 centimeters, 8 to 12 centimeters, and 12 to 18 centimeters long for 1, 2, and 3 level posterior lateral fusions, respectively. Longer heat emitting portions are used for four or more level fusions. Alternatively, more than one heat emitting elements 15, arranged serially or longitudinally, but not necessarily connected, could be used for fusions of two or more levels. Each such heating element 15 is preferably connected to separate channels of a temperature controlling unit (see below). Heating elements 15 may also be placed parallel to one another on the same side of the spine. For example, heating elements 15 could preferably be placed 0.5 cm to 6 cm or more apart. Parallel heating elements 15 may be used to heat large volumes of tissue or synthetic materials to the desired temperature with small diameter heating elements, which are easily pulled from the wound at the end of heating. For example, one heating element 15 could preferably be used per 50 mm² to 400 mm² area of tissue or synthetic material in cross-section. So, for example, 2 to 14 ten cm long heating elements 15 could preferably be used to heat a 3 cm wide by 10 cm long volume of tissue. Using multiple heating elements 15 in a single patient enables a combined cross-sectional surface area of heating elements of 80 mm² to 120 mm² or more to be created with a plurality of heating elements 15, each smaller than 4 mms in diameter. The invention enables precise targeting of the volume of tissue or material to be heated. For example, targeted areas as small as 0.5% to 5% of the cross-sectional area of the body can be heated in a controlled manner. The invention also enables heating of precise volumes of tissue or material. For example, heating may be limited to volumes of tissue or material as small as 140 mm³ or smaller to 39° C. to 41.9° C. Generally the volume of heated tissue or material heated is within 60% to 140% of the intended volume of tissue to be heated. The invention enables precise control of maximum and average temperatures of the tissues being heated. Generally the maximum temperature of the heated tissue is 41.9° C. and the average temperature of the heated tissue or material is 40.5° C. to 41.4° C.±0.5° C. Such precision enables controlled heating of tissue or material which may lie adjacent to vital structures, such as the spinal cord, whereby to permit the desired heating of the target tissues without harming the adjacent vital structures, and structures or materials that shunt heat, such as the aorta, vena cava, spinal fluid, and metal rods, screws, and plates, whereby to permit heating the target tissues to the desired temperature even when the target tissues significantly shunt heat. The present invention avoids heating non-targeted tissues, thus preventing macrophage attraction to those non-targeted tissues.

Transplanted autograft bone contains living osteogenic cells and leukocytes. Macrophages in such living tissue and the adjacent bones react to heat by releasing cytokines, which attracts inflammatory cells, such as neutrophils and macrophages, to the heated tissue. Such inflammatory cells initiate the normal healing process. Macrophages, specifically activated macrophages, are essential to normal tissue healing. The invention, by supplying carefully controlled heat to target tissues, attracts more inflammatory cells, especially activated macrophages, to the target tissues (e.g., autograft bone and the adjacent vertebrae) than would normally occur through natural body processes. The invention also attracts macrophages to such living tissues sooner than would normally occur through natural body processes. So the invention accelerates and augments natural healing of transplanted autograft bone, i.e., by supplying carefully controlled heat to target tissues, whereby to attract more inflammatory cells, especially activated macrophages, to the target tissues.

Allograft bone and synthetic bone substitutes contain no living cells, so heating these tissues does not directly promote a healing process within those tissues per se. However, heating such tissues heats adjacent living tissues, such as muscle and the vertebrae. Macrophages in those living tissues then react to the supplied heat to release cytokines, which attract macrophages to the heated living tissues and to the adjacent autograft bone substitutes. Thus, the invention makes osteoconductive-only materials, such as hydroxyapatite (ProOsteon, Biomet, Parsippany, N.J.), collagen-based matrices, calcium phosphate, calcium sulfate (Osteoset, Wright Medical technology, Arlington, Tenn.), and bioactive glass, effectively osteoinductive. The invention also makes weakly osteoinductive materials, such as demineralized bone matrix (AlloGro, AlloSource, Centennial, Colo.), more osteoinductive. Autogenic cells, such as those found in small pieces of autograft bone, platelet rich plasma (PRP), the “buffy coat” of centrifuged blood, or bone marrow aspirates, may preferably be added to those bone graft substitutes without living cells, in addition to the application of heat, so as to promote healing. Macrophages from such sources, or monocytes from such sources which become macrophages, respond to heating of those bone graft substitutes in the previously mentioned fashion, which recruits additional macrophages to the bone graft substitute, whereby to promote healing.

Heating the bone growth materials and the surrounding tissues attracts macrophages and activates them. Human macrophages are optimally attracted to, and activated by, tissues heated to 40° C. to 41° C. for at least forty-eight hours. The invention preferably heats the healing tissues and bone growth materials to 38° C. to 41.9° C., more preferably to 39° C. to 41.9° C., and most preferably to 40° C. to 41° C. The invention limits maximum temperatures to 41.9° C. because heating human tissue to 42° C. for thirty minutes kills cells in that tissue. The invention preferably heats tissues and bone graft materials for several hours to about two weeks, more preferably for ten hours to seven days, and most preferably for one to three days, in order effect desired healing. Bone growth materials, without living cells, may preferably be heated to more than 42° C. in order to heat the living tissues adjacent to such materials to the desired temperature of about 40° C. to 41° C. However, such bone growth materials are preferably heated to less than 45° C., so as to not harm the adjacent living cells.

Joule heating from electric resistors or other elements with electrical resistance at the distal end of the heating element 15 preferably heats the tissues and, where applicable, synthetic materials or devices. For example, the distal end of the heating element 15 preferably contains a high resistance wire, such as Nichrome or a biocompatible material, that is preferably coiled around or embedded in a synthetic electrical non-conducting material that has very good heat absorption and emission characteristics, such as a ceramic. The loops of the wire coil are preferably 0.1 mm to 2.0 mm apart. The synthetic non-conducting material preferably extends between the loops of the wire coil so as to prevent short circuits along the wire coil. For example, a cartridge heater (McMaster-Carr, Aurora, Ohio) could be located at the distal end of the heating element 15. The heating element 15 is made of biocompatible materials, well known to those skilled in the art of cardiac pacemakers, cardiac defibrillators, dorsal column stimulators, and cochlear implants. The heating elements 15 could preferably be straight or curved and they may be sealed to prevent contact with body fluids. Methods of sealing electrical implants are well known to those skilled in the art of cardiac pacemakers, cardiac defibrillators, dorsal column stimulators, and cochlear implants. Flexible cartridge heaters, for example with a heater having flexible sections or joints between ridged sections, are especially preferable. The heating section of the heating element 15 is preferably about 0.5 cm to 40 cm in length, depending on the application. For example, the heating section could be supplied in 0.5, 1, 2, 3, 4, 5, 8, 11, 15, 20, 25, 30, 35, & 40 cm lengths. The proximal portion of the heating element 15 is preferably insulated and flexible. Such portion of the heating element 15 is preferably 25 to 100 cm long. A trocar, which cuts the skin, may be reversibly fastened to the proximal end of the heating element. The heating element 15 is preferably about 1 to 10 millimeters in diameter, more preferably about 3 to 8 millimeters in diameter, and most preferably about 3 to 5 millimeters in diameter. The proximal end of the heating element 15 is connected to a temperature control unit (see below) such as a Single Channel Temperature Control Panel (OEM Supply Inc., Swansea, Mass.). More preferably the temperature control unit has two to ten or more channels, with each channel being capable of separately controlling a heating element 15. The heating element 15 preferably has a temperature sensor, the distal end of which is placed in the heated tissue and the proximal end of which is connected to the temperature control unit, to ensure precise temperature control, preferably with an accuracy of ±0.5° C. or less. The heating element 15 could use alternative heat generating features in alternative embodiments of the invention. For example, one or more high resistance wires could be used rather than cartridge heaters, or circulating heated fluid could pass through the heating element, or an external ultrasound (US) or radiofrequency (RF) generator could heat an internal component, or a fully external US, RF, IR or other type of unit could heat the tissues.

FIG. 1B is a superior view of a partial transverse cross section of the spinal segment, bone growth material, and the embodiment of the invention shown in FIG. 1A. The heating elements 15 are seen centrally located in the bone growth material 10. Muscles 25 are seen superior or dorsal to the spine 5 and bone growth material 10. Two, three, four or more heating elements could be inserted into, and/or adjacent to, the bone growth material 10 in alternative embodiments of the invention. Each such heating element 15 is preferably connected to separate channels of a temperature control unit. Alternatively, each heating element 15 could be connected to separate temperature control units. The invention heats the bone growth material, and preferably the vertebrae and muscles, to a carefully controlled temperature, whereby to attract and activate the inflammatory cells which promote healing. For example, the bone growth material and surrounding tissues could preferably be heated to 40° C. to 41° C. for 12 to 48 hours following surgery, preferably starting immediately following surgery, whereby to enhance the body's natural healing processes.

FIG. 2A is a posterior view of a portion of the spine 5, bone growth material 10 and an alternative embodiment of the invention. High resistance wires 30 of the two heating elements 15 are seen coiled around the bone growth material 10. The proximal ends 35 of the heating elements 15 are preferably insulated and are connected to a temperature control unit (not shown). Temperature sensing elements, not shown, are preferably placed near the bone growth material 10 and the adjacent tissue. The proximal ends of such temperature sensing elements are connected to the aforementioned temperature control unit. The temperature sensing elements are preferably placed against or within 2 centimeters of the heating elements so as to permit precise regulation of the temperature of the heating elements, and hence precise regulation of the temperature of the target tissues. A releasable connection or couple (not shown) just proximal to the distal heating portion of the heating element 15 could, optionally, enable the proximal portion of the heating element to be pulled from the body while leaving the heating portion of the heating element in the body.

FIG. 2B is a superior view of a partial transverse cross-section of the spinal segment 5, bone growth material 10, and the heating elements 15 shown in FIG. 2A.

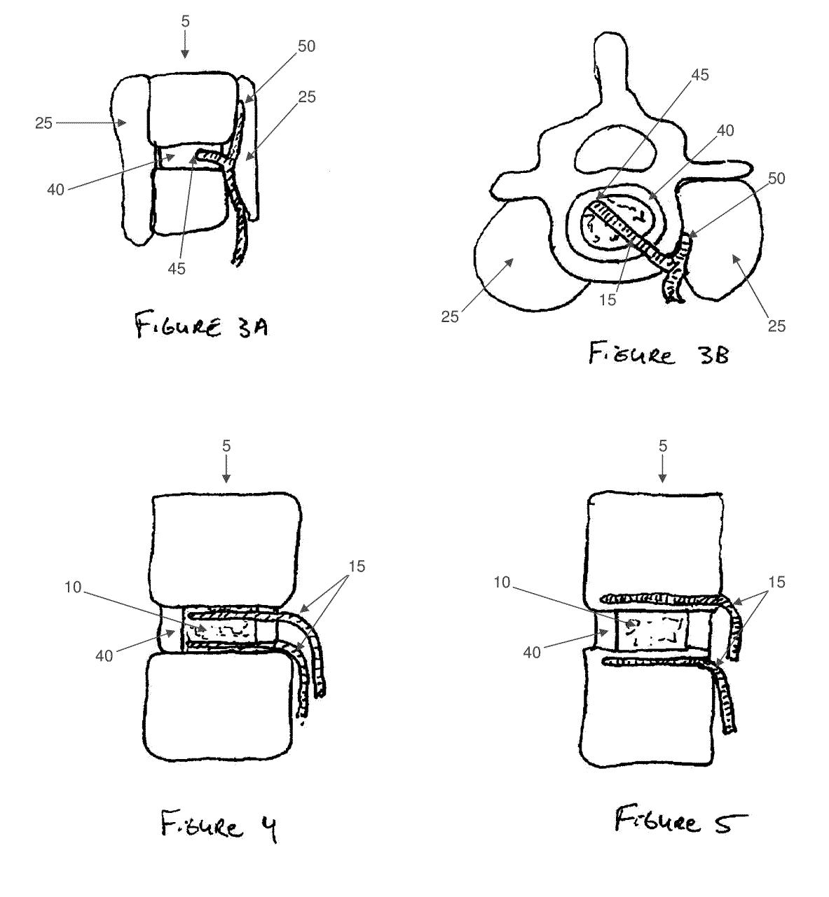

FIG. 3A is an anterior view of a portion of the spine 5, an intradiscal fusion device (e.g., a cage or disc replacement) 40, and an alternative embodiment of the invention. A first distal end 45 of the heating element 15 is seen passing through an opening in the cage or disc replacement 40. A second distal end 50 of such element is seen between the lateral aspect of the spine and the overlying muscles 25. Thus, in this form of the invention, heat is applied by distal end 45 of heating element 15 to the material located within cage 40, and to the vertebral body and overlying muscles by second distal end 50 of heating element 15. Heat may also be applied to other tissues adjacent to other portions of heating elements 15. The distal portion of the heating element could have 3, 4, 5, 6, 7, 8, 9, 10, 11, 12, 13, 14, or more distal heat-emitting features in alternative embodiments of the invention. Wires from each heat-emitting feature are preferably connected to separate channels in the temperature control unit. Alternatively, 2, 3, 4, 5, 6, 7, 8, 9, 10, 11, 12, 13, 14, or more separate heating elements could be used in other embodiments of the invention. The volume of tissue or synthetic material heated by one distal heating element can preferably be different in size, shape, or orientation than such volume of tissue or synthetic material heated by a second heating element in the same patient. For example, the end 45 of the heating element 15 seen passing into the intradiscal cage 40 heats a volume of tissue or material that is generally smaller and perpendicular to the volume of tissue heated by the portion 50 of the heating element 15 that lies against the side of the spinal segment.

FIG. 3B is a superior view of a partial transverse cross-section of the spinal segment 5, bone growth material 10, intradiscal fusion device (e.g., a cage or disc replacement) 40 and the embodiment of the invention shown in FIG. 3A. The invention preferably heats the bone growth material 10, at least one of the vertebral endplates of the adjacent vertebrae, at least the sides of the vertebral bodies, and the muscle 25. The invention appropriately heats the macrophages in such living tissue, which attracts inflammatory cells to such tissues and to the bone growth material. The heating elements 15 preferably heat at least some living tissue, which contains many living macrophages. Such living tissues, such as muscle, bone, and organs, generally have a relatively high number of blood vessels per volume of tissue. The distal end of a heating element 15 may be placed adjacent to a cage or prosthetic disc replacement, or adjacent to the adjacent vertebrae, to promote bone ingrowth into the prosthesis in alternative embodiments of the invention. Similarly, distal ends of heating elements 15 may be placed adjacent to other prosthetic joints, such as hips, knees, shoulders, and ankles, or adjacent to bones in which the prostheses are implanted, in alternative embodiments of the invention, so as to promote bone growth into such prostheses.

FIG. 4 is an anterior view of a partial coronal cross-section of a spinal segment 5, intradiscal device 40, bone growth material 10 and an alternative embodiment of the invention. Separate heating elements 15, or the separate ends of a single heating element, are seen coursing through holes near the top and bottom of the intradiscal device 40 and the bone growth material 10. This embodiment of the invention is particularly good for heating at least both vertebral endplates and the bone growth material.

FIG. 5 is an anterior view of a partial coronal cross-section of a spinal segment 5, intradiscal device 40, bone growth material 10 and an alternative embodiment of the invention. Separate heating elements 15, or the separate ends of a single heating element, are seen passing through holes drilled through the vertebral bodies near the vertebral endplates. Such holes are preferably within 1 to 10 millimeters of the endplates. Distal ends of heating elements 15 may be placed in such locations adjacent to a prosthetic intradiscal device to promote bone ingrowth into the prosthesis in alternative embodiments of the invention. Similarly, distal ends of heating elements 15 may be placed into bones in which prosthetic joints, such as hips, knees, shoulders, and ankles, are implanted in alternative embodiments of the invention, so as to promote bone growth into such prostheses. If desired, small elastic projections (not shown) could extend from the sides of the distal end of the heating element 15. These projections expand into the vertebrae to help hold the heating elements in the vertebrae. Alternatively, the distal end of the heating element 15 could be radially expanded in order to temporarily hold a heating element in position in alternative embodiments of the invention. For example, high resistance wire could be wrapped around or embedded in a balloon. The balloon is inserted (in deflated condition) into the holes formed in the vertebral body and then expanded in the vertebral body so as to hold the heating wire in position and to increase the surface area of the heating device. The balloon is collapsed and the device pulled from the body after the desired heat treatment is applied.

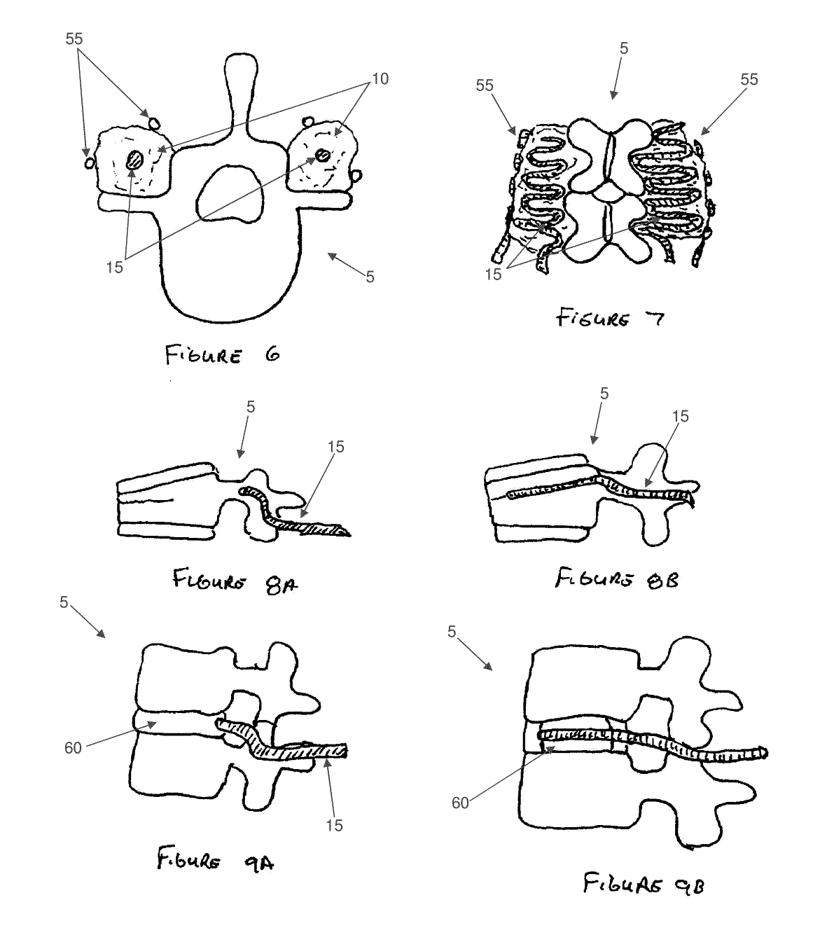

FIG. 6 is a superior view of a transverse cross-section of the spinal segment 5, bone growth material 10, and an alternative embodiment of the invention. Two or more temperature-sensing components 55 are seen on each side of the spine, between the bone growth material 10 and the surrounding soft tissues. Single heat-emitting components 15 are seen on each side of the spine and in the bone growth material. Three, 4, 5, 6, 7, 8, or more temperature-sensing components 55 could be used in alternative embodiments of the invention. Two, 3, 4, 5, 6, 7, 8, or more heat-emitting components 15 could be used in alternative embodiments of the invention. The proximal ends of the heat-emitting components 15 and temperature-sensing components 55 are connected to a temperature control unit. The temperature control unit adjusts the electricity sent to the heating components 15 and thus regulates the temperature of those heating components based upon input from the temperature-sensing components 55. The temperature control unit preferably has one or more additional safety features to prevent excessive temperatures. For example, the temperature control unit is preferably shut off when the unit loses input from one or more temperature sensing elements. Furthermore, electric output from the unit may be limited based upon the tissue to be heated. For example, the average number of watts from the unit needed to heat a lumbar IVD to 40.5° C. in cadavers, or more preferably patients, is determined. Such average number is multiplied by 1.1 and that product is used as a maximum allowable output number when using the invention to heat IVDs. Alternatively, the average electrical output required to heat a lumbar IVD to 42° C. can be determined in cadavers or patients. Such average number is multiplied by 0.9 and that product is used as a maximum allowable output number when using the invention to heat IVDs. Such methods and maximum allowable outputs are determined for each type of tissue to be heated As noted above, heating elements 15 placed in living tissue are preferably limited to a precisely controlled maximum temperature of 41.9° C. in order to promote the desired inflammatory response without killing living cells. The embodiment of the invention is particularly good for heating the bone growth material 10, which lacks living cells, to a temperature above 41.9° C. so as to heat the surrounding living tissue to a temperature which promotes the healing response without killing living cells, for example 40° C. to 41.5° C.

FIG. 7 is a posterior view of a spinal segment 5, bone growth material 10, and an alternative embodiment of the invention. Two heating elements 15 are seen on each side of the spine. The serpiginous or serpentine (snake-like) shape of the heating elements 15 increases the surface area of the heated tissue.

FIG. 8A is a lateral view of a spinal segment 5 and an alternative embodiment of the invention. The distal end of a heating element 15 is seen passing into a hole drilled into a fractured vertebra. The hole is preferably drilled through the pedicle of the vertebra percutaneously, under local anesthesia, with a trocar and an outer cannula of the sort well known in the orthopedic arts. The distal end of the heating element 15 is passed through the cannula after removing the trocar. The invention carefully heats the vertebra, especially near the fractured portion of the vertebra, so as to increase the inflammation response of the tissue, which accelerates healing of the fracture. For example, fractured vertebrae could preferably be heated to 40° C. to 41° C. for 12 to 48 hours, whereby to promote an inflammatory response and thereby enhance healing. Alternatively, the vertebrae could be heated to 42° C. to 45° C. for 20 to 60 minutes to kill nerve cells, which at least temporarily decreases pain from the vertebra, then the temperature could be lowered to 40° C. to 41° C. for 12 to 72 hours to promote an inflammatory response and accelerate healing of the fracture. The embodiment of the invention could also be used to treat infection or tumors in vertebrae. Heating vertebrae attracts leukocytes to the vertebrae and activates the leukocytes. Activated leukocytes kill bacteria and/or kill tumor cells better than non-activated leukocytes. Particularly when treating tumors, the invention could first heat the vertebrae above 42° C. to kill tumor cells, then heat the treated tissue to, for example, 40° C. to 41.5° C. for 12 to 72 hours or more to increase inflammation and hence promote healing. The embodiment of the invention could be used to treat other bones in the body. In alternative embodiments of the invention, cannulated heating elements could be passed over guidewires so as to deliver the heating elements to target tissue in hard-to-reach locations and/or via “keyhole” (e.g., arthroscopic) surgery. Such embodiments of the invention facilitate percutaneous heating element insertion, increase the diameter of the heating elements, which increases the volume of heated tissue, and may enable the heating elements to collapse as they are pulled through small incisions in the skin.

FIG. 8B is a lateral view of a partial sagittal cross-section of the spinal segment and embodiment of the invention shown in FIG. 8A. The heat-emitting portion of the heating element 15 is preferably about 2 cm to about 4 cm long.

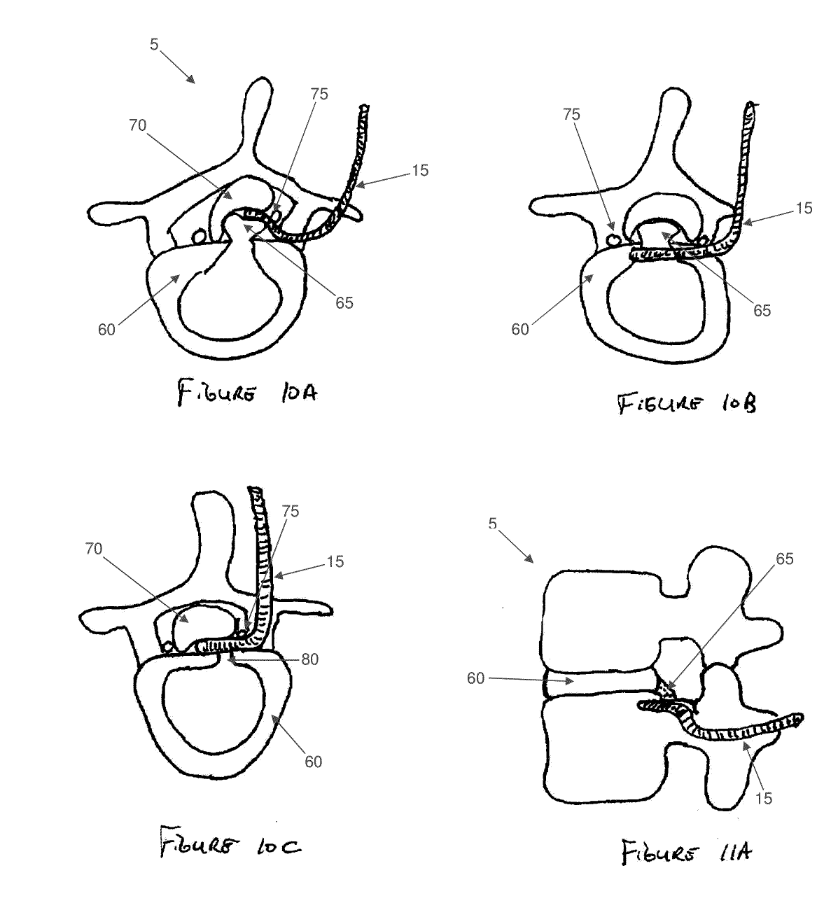

FIG. 9A is a lateral view of a spinal segment and an alternative embodiment of the invention. The distal end of the heating element 15 is seen passing into a hole formed in the intervertebral disc (IVD) 60. The heating element 15 preferably heats at least a portion of the IVD to 38° C. to 41.8° C., without heating any portion of the IVD above such temperatures, for preferably several hours to several days and, most preferably, to 40° C. to 41° C. for 12 to 72 hours. The embodiment of the invention is used to treat disc infections, HNP, and disc degeneration. Fluid, such as saline, PRP, leukocytes in the Buffy coat of centrifuged blood, or bone marrow aspirate, could be added to the IVD. Such fluid, especially viscous fluid or PRP gel, helps distribute the heat throughout the IVD. This embodiment of the invention, including fluid injection, could be used to treat arthritis in joints, such as hips, knees, shoulders, ankles and wrists. For example, heating elements are preferably placed between the skin and the joint capsule of the anterior, medial, lateral, and posterior aspects of the knee to treat arthritis, osteonecrosis, inflammation or other knee pathology. Such heating elements are preferably placed near the superior lateral, inferior lateral, and superior medial genicular arteries and near lesser branches of the popliteal artery. Alternatively, such heating elements could be placed intra-articular or intra-osseous in other embodiments of the invention. Heating elements are preferably placed in the subacromial space to treat bursitis, tendonitis and rotator cuff tears of the shoulder. The heat-emitting portion of the heating element 15 is preferably about 2 cm to about 4 cm long. The distal end of the heating element 15 is preferably inserted into the IVD using the trocar and cannula approach taught above with respect to the form of the invention shown in FIG. 8A.

FIG. 9B is a lateral view of a partial sagittal cross-section of the spinal segment 5 and the embodiment of the invention shown in FIG. 9A.

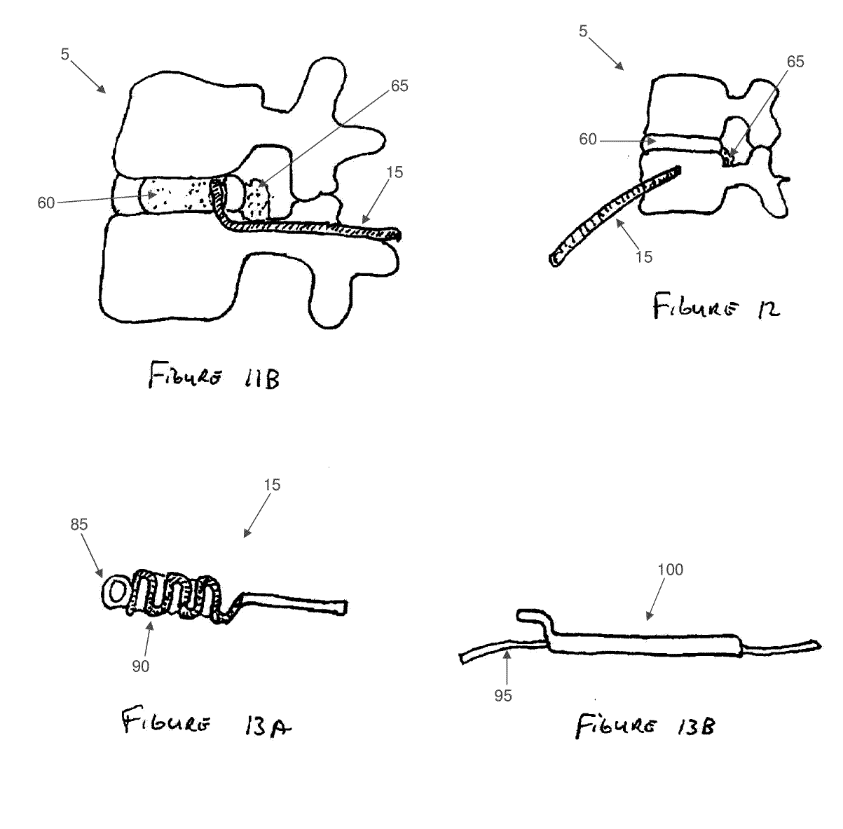

FIG. 10A is a superior view of a partial transverse cross-section of a spinal segment 5 and an alternative embodiment of the invention. NP tissue 65 is seen extruding through an aperture in the anulus. Such NP tissue 65 is seen compressing the thecal sac 70 and a small nerve 75. The distal end of a heating element 15 is seen between the HNP 65 and the thecal sac 70 and the compressed nerve 75. The heating element 15 preferably heats the HNP 65, and possibly the surrounding tissue, to 38° C. to 41.5° C., more preferably to 39° C. to 41° C., and most preferably to 40° C. to 41° C., for several hours to several days, and more preferably for 1 to 3 days. Such heating attracts macrophages to the HNP, and activates the macrophages, to accelerate removal of the HNP. The heating element 15 may be positioned between HNP 65 and the thecal sac 70 and compressed nerve 75, in the following manner. The distal end of a guidewire (not shown) is preferably directed over the HNP in the first step of the procedure. Such guidewire is preferably passed through a straight or curved needle of the sort well known in the art. A tissue dilator may be passed over the guidewire in the next step of the procedure, and then a cannulated heating element is passed over the guidewire and into the body. Alternatively, a tissue dilator and cannula can be passed over the guidewire, and then the distal end of the heating element is passed through the cannula after removing the guidewire and tissue dilator. The procedure is preferably performed under local anesthesia. It should be appreciated that the enhanced macrophage-mediated HNP removal of the present invention is in sharp contrast to prior art epidural steroid injection treatments of HNP, which decrease inflammation (and hence retard macrophage-mediated HNP removal). The distal end of the heating element 15 is preferably passed into the HNP in alternative embodiments of the invention. In such embodiments of the invention, the HNP may be temporarily heated to 42° C. or above to kill HNP cells before treatment at lower temperatures to increase inflammation and hence HNP removal. For example, the HNP could be heated to 42° C. to 45° C. for 15 to 60 minutes to kill HNP cells, then the HNP could be heated to 40° C. to 41° C. for 1 to 2 days for HNP removal. The heat-emitting portion of the heating element 15 is preferably about 1 cm to about 4 cm long. The distal end of the heating element 15, or a second heating element, could be passed from the opposite side of the spine, between the non-compressed nerves and the IVD 60, in an alternative embodiment of the invention. The thecal sac side of the heating element could be insulated with a material, such as plastic, to prevent heating the nerves in an alternative embodiment of the invention.

FIG. 10B is a superior view of a partial transverse cross-section of a spinal segment and an alternative embodiment of the invention. The distal end of the heating element 15 is seen passing through a hole in the IVD 60 and across NP tissue 65. The embodiment of the invention places the heating elements 15 away from the nerves, relative to the embodiment of the invention shown in FIG. 10A where the heating elements 15 are placed adjacent to the nerves. The form of the invention shown in FIG. 10B may, advantageously, protect the nerves, especially if temperatures above 42° C. are used.

FIG. 10C is a superior view of a partial transverse cross-section of a spinal segment and an alternative embodiment of the invention. The distal end of the heating element 15 is seen between a portion of the thecal sac 70, a nerve 75 and an aperture 80 in the AF. The heating element 15 could be placed in such location following surgical discectomy to accelerate removal of any additional residual extruded NP tissue or any additional NP tissue that extrudes through the aperture 80 in the first few days following discectomy.

FIG. 11A is a lateral view of a spinal segment and an alternative embodiment of the invention. The distal end of the heating element 15 is seen passing into a hole in the posterior portion of the vertebral body. Extruded NP tissue 65 is seen extending from the IVD 60 and behind the lower vertebra. The invention heats the lower vertebra when NP tissue 65 extends behind that vertebra and the upper vertebra when NP tissue 65 extends behind that vertebra. The invention heats at least the posterior portions of vertebrae and preferably portions of the IVD 60, HNP 65, and the posterior longitudinal ligament.

FIG. 11B is a lateral view of a partial sagittal cross-section of the spinal segment and the embodiment of the invention shown in FIG. 11A. The distal end of the heating element 15 is seen passing through a vertebral endplate and into the IVD 60 anterior to the HNP 65. Alternatively the distal end of the heating element 15 could remain in the vertebra. The distal end of the heating element 15 could be placed into the upper vertebra in an alternative embodiment of the invention.

FIG. 12 is a lateral view of a spinal segment and an alternative embodiment of the invention. The distal end of the heating element 15 passes through a hole in the lateral portion of the vertebra and extends to the posterior portion of the vertebra and possibly into the IVD 60 or spinal canal. Alternatively the distal end of the heating element 15 could be passed through a hole in the anterior portion of the vertebra. The distal end of the heating element 15 could pass through a hole in the upper vertebra in an alternative embodiment of the invention.

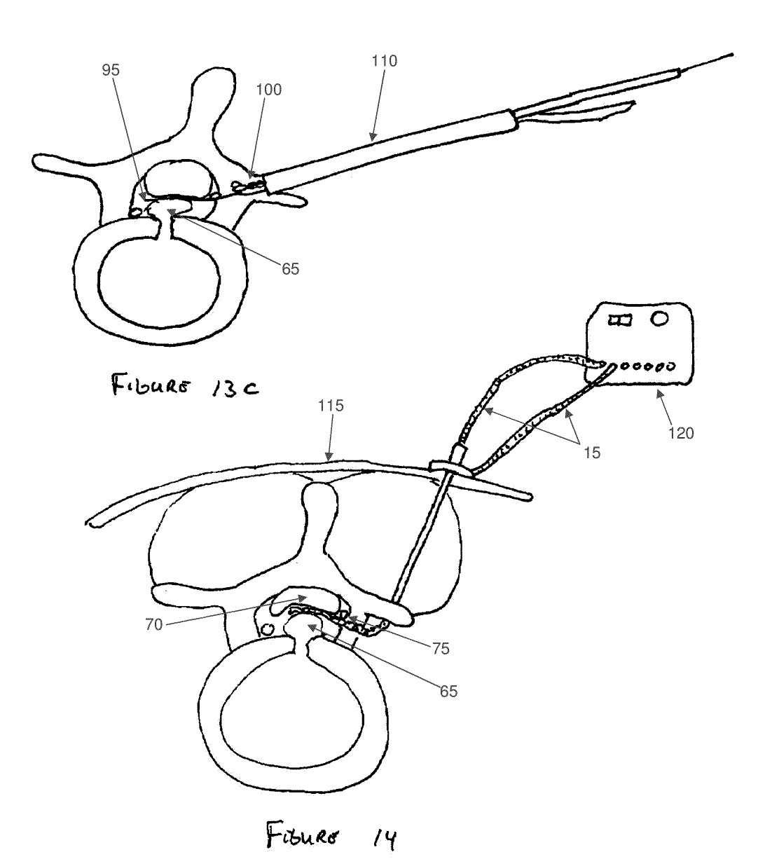

FIG. 13A is a superior view of the distal end of an alternative embodiment of the invention. A loop 85 is seen formed at the distal end of the heating element 15. A snake-like portion 90 of the heating element 15 is seen proximal to the loop 85. The snake-like portion 90 of the heating element 15 may be embedded in or fastened to a flexible, preferably plastic or non-electric-conducting material, sheet. Alternatively, tension bands (not shown) could extend between the curved sections of the resistive wire which is used in heating element 15.

FIG. 13B is a lateral view of additional novel tools which may be used in connection with the embodiment of the invention shown in FIG. 13A. A guidewire 95 is seen passing through a cannulated insertion tool 100. The guidewire 95 is preferably about 1 mm to 2 mm in diameter and about 30 cm to 50 cm long. The internal diameter of the insertion tool 100 is slightly larger than the diameter of the guidewire 95. The insertion tool 100 is preferably about 10 cm to 25 cm long. The guidewire 95 and insertion tool 100 are preferably made of metal, such as stainless steel or nitinol. Alternatively, the insertion tool 100 could preferably be made of plastic.

FIG. 13C is a superior view of a partial transverse cross-section of a spinal segment, and the embodiments of the invention shown in FIGS. 13A and 13B. The distal end of the guidewire 95 is placed near the HNP 65 in the first step of the procedure. A tissue dilator (not shown) and a cannula 110 are passed over the guidewire 95 in the next step of the procedure, then the tissue dilator is removed. The loop on the distal end of the heating element 15 is passed over the curved distal end of the insertion tool 100, then the insertion tool 100 is passed over the guidewire 95 and through the cannula 110. The distal end of the heating element 15 is pushed to the HNP 65, then the guidewire 95, insertion tool 100, and cannula 110 are removed. Alternatively, additional heating elements could be pushed to the HNP before the cannula is removed.

FIG. 14 is a superior view of a partial transverse cross-section of human body, including a spinal segment and the alternative embodiment of the invention shown in FIG. 13C. A second heating element 15 is seen surrounding at least a portion of a first heating element 15 where the first heating element 15 passes through the skin. The distal end of the second heating element 15 is preferably fastened to the skin 115 with adhesive. The distal end of the first heating element 15 is seen between the HNP 65 and the thecal sac 70 and a nerve 75. The second heating element 15 heats the skin 115 around the middle portion of the first heating element 15. The invention increases inflammation in the skin around the middle portion of the first heating element, which attracts bacteria-killing leukocytes to that area, which decreases the risk of infection. The proximal ends of the heating elements 15 are seen connected to a multi-channel temperature control unit 120. The embodiment of the invention could be used to prevent infection of other devices that pass through the skin, such as intravenous catheters and pins of external fixators, which hold bones in alignment or apply forces to bones. The flexible proximal portion of the heating element 15 easily bends, enabling patients to comfortably lie on the wires. The flexible proximal portion of the heating element 15, which extends through the skin or passes near the skin, also allows movement of such portion of the heating element 15 relative to the skin without fear of moving the distal end of the heating element 15.

The invention can be used in all tissues of the body. For example, a heating element, such as a resistor wire, could be coiled around a urinary catheter. Such heating element could be used to heat the prostate to about 40° C. to 41.9° C. for one to three days or more. Such embodiment of the invention could be used to treat prostatitis, benign prostatic hyperthrophy, or prostatic cancer. Catheters with heating elements could be coiled in the urinary bladder to treat interstitial cystitis and urinary tract infections. Such catheters could also be passed into the kidney, preferably through the ureter to treat pyelonephritis. Heating elements could also be placed in the vagina to heat the vaginal cuff, vagina, and surrounding tissue following gynecologic procedures.

### Modifications of the Preferred Embodiments

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. Apparatus for treating tissue, wherein the apparatus heats the tissue in a controlled manner so as to promote therapeutic inflammation in the tissue, whereby to augment healing of the tissue.

2. Apparatus according to claim 1 wherein the tissue is heated in a controlled manner so as to attract neutrophils so as to promote the normal healing inflammatory process, which at its conclusion recruits reparative cells and drives away inflammatory cells.

3. Apparatus according to claim 1 wherein the neutrophils comprise macrophages, the reparative cells comprise fibrocytes and the inflammatory cells comprise lymphocytes.

4. Apparatus according to claim 1 wherein the tissue is heated to 38° C. to 41.9° C. for a period of several hours to two or more weeks.

5. Apparatus according to claim 4 wherein the tissue is heated to 39° C. to 41.9° C. for a period of ten hours to one week.

6. Apparatus according to claim 5 wherein the tissue is heated to 40° C. to 41° C. for a period of one to three days.

7. A method for treating tissue, comprising:
heating the tissue in a controlled manner so as to promote therapeutic inflammation in the tissue, whereby to augment healing of the tissue.

8. A method according to claim 7 wherein the tissue is heated in a controlled manner so as to attract neutrophils so as to promote the normal healing inflammatory process, which at its conclusion recruits reparative cells and drives away inflammatory cells.

9. A method according to claim 7 wherein the neutrophils comprise macrophages, the reparative cells comprise fibrocytes and the inflammatory cells comprise lymphocytes.

10. A method according to claim 7 wherein the tissue is heated to 38° C. to 41.9° C. for a period of several hours to two or more weeks.

11. A method according to claim 10 wherein the tissue is heated to 39° C. to 41.9° C. for a period of ten hours to one week.

12. A method according to claim 11 wherein the tissue is heated to 40° C. to 41° C. for a period of one to three days.
